(19) United States
(12) Patent Application Publication
(10) Pub. No.: US 2025/0228442 A1
(43) Pub. Date: Jul. 17, 2025

(54) ENDOSCOPE

(71) Applicant: CMR Surgical Limited, Cambridge (GB)

(72) Inventor: Roger James Springett, Northamptonshire (GB)

(73) Assignee: CMR Surgical Limited, Cambridge (GB)

(21) Appl. No.: 19/021,872

(22) Filed: Jan. 15, 2025

(30) Foreign Application Priority Data
Jan. 15, 2024 (GB) 2400548.0

(51) Int. Cl. A61B 1/04; A61B 1/00; A61B 1/05; A61B 1/06; A61B 1/07

(52) U.S. Cl. CPC A61B 1/043; A61B 1/00096; A61B 1/05; A61B 1/0627; A61B 1/063; A61B 1/0653; A61B 1/07

(57) ABSTRACT

An endoscope and a method of operating an endoscope are provided. The endoscope includes a set of one or more optical fibres connecting a proximal end of the endoscope to a distal end of the endoscope. The set of one or more optical fibres is arranged to receive, at the proximal end of the endoscope, light generated by a light source. The endoscope also includes a phosphor arranged at the distal end of the endoscope to receive light from the set of one or more optical fibres and emit light having a different spectrum to the light from the light source.

### CROSS-REFERENCE TO RELATED APPLICATION

This application claims priority to GB Patent Application No. 2400548.0, filed Jan. 15, 2024, which is incorporated herein by reference in its entirety.

### BACKGROUND

An endoscope is an inspection instrument, which may be used during minimally invasive surgery. An endoscope can be inserted into a body of a patient through an opening, which may be a natural orifice such as the mouth, or may be through a port to access a surgical site during surgery. An endoscope typically comprises a camera and an interface for connecting to a light source, and is typically used to illuminate and image a surgical site for a surgeon during minimally invasive surgery. A video feed from the camera is displayed on a surgeon's display during surgery to enable the surgeon to view the surgical site. Some endoscopes have a rigid body, while some other endoscopes have a flexible body. Typically, the imaging quality is higher for a rigid endoscope compared to a flexible endoscope, whereas a flexible endoscope is typically more manoeuvrable than a rigid endoscope so may be able to obtain a better viewpoint for viewing the surgical site.

An endoscope is an elongate instrument, which comprises a proximal end and a distal end. The proximal end of the endoscope is not intended to be inserted into a patient, whereas the distal end of the endoscope is intended to be inserted into a patient, e.g. during surgery. FIG. 1 illustrates an arrangement for coupling light from a light-emitting diode (LED) 102 and a phosphor 104 into an optical fibre 106 for use in a conventional endoscope. The LED 102 and the phosphor 104 may have any suitable shape, but usually high power LEDs are planar, with the phosphor coated on top, as shown in FIG. 1.

An LED is a semiconductor device that emits light when current flows through it. The colour of the light emitted from an LED is determined by the energy required for electrons to cross the band gap of the semiconductor. As such, most LEDs emit light within a narrow range of wavelengths, such that they have a strong characteristic colour. However, often, it is desirable for an endoscope to illuminate a surgical site with light that appears substantially white.

Illuminating the surgical site with white light tends to be most useful for a surgeon viewing the images of the surgical site provided by the endoscope. White light can be obtained using a layer of light-emitting phosphor on the LED.

A phosphor is a substance that exhibits the phenomenon of luminescence. That is, a phosphor emits light when exposed to some type of radiant energy. Phosphors are designed to absorb and emit at certain wavelengths and those that operate to absorb blue light, e.g. with wavelengths in the 440-455 nm range, are very efficient in terms of the ratio between the radiant power that they can output and the radiant power that they receive. As such, conventional white LEDs tend to generate blue light and use a phosphor to convert the blue light to white light.

Referring to FIG. 1, the LED 102 is a blue LED, and is coated with the phosphor 104 which is configured to absorb some of the blue light emitted from the LED 102 and emit green and red light. Some of the blue light emitted from the LED 102 passes through, or is scattered by, the phosphor 104. The resulting combination of blue, green and red light coming from the arrangement of the LED 102 and the phosphor 104 appears as white light.

The optical fibre 106 is a cylindrical dielectric waveguide that transmits light along its axis through the process of total internal reflection. FIG. 1 shows the end of the optical fibre 106 being located close to the arrangement of the LED 102 and the phosphor 104. The LED is a large area emitter. The radial lines in FIG. 1 around the arrangement of the LED 102 and the phosphor 104 represent the light being radiated over a large area. Only some of the light emitted from the LED 102 and phosphor 104 is received by the optical fibre 106. A lens may be used to focus the light emitted from the LED 102 and phosphor 104 onto the optical fibre 106, but it is still the case that a significant proportion of the light emitted from the LED 102 and phosphor 104 is not received by the optical fibre 106. Furthermore, the optical fibre 106 will only accept light over a much smaller cone of angles than is emitted from the LED. As such, coupling light from the LED 102 into the optical fibre 106 is inefficient, i.e. a significant proportion of the radiant energy that is emitted from the LED 102 does not travel down the optical fibre 106. This can cause a problem when LEDs are used with an endoscope for performing endoscopy, e.g. for minimally invasive surgery. For example, in order to pass enough light into the optical fibre 106 to produce sufficient illumination of the tissue at the surgical site, high-power LEDs are required, and a set (or ‘bundle’) of one or more optical fibres with a large diameter is required. For example, a conventional endoscope for endoscopy may comprise a bundle of optical fibres with a diameter in a range from 3 mm to 5 mm. Such a large bundle of optical fibres is heavy, large and lacks flexibility. It is generally beneficial for instruments that enter a patient's body during minimally invasive surgery to be light, flexible (i.e. manoeuvrable) and small (in particular to have a small cross-section, e.g. diameter).

FIG. 2 illustrates a distal end of a conventional endoscope, which comprises an optical fibre 206. Light exits the distal end of the optical fibre 206 within a cone, as illustrated by the arrows coming out of the top of the optical fibre 206 in FIG. 2. The distal end of the endoscope may also comprise a camera (not shown in FIG. 2) to capture images of the surgical site. In some endoscopes, a camera is located at the proximal end of the endoscope rather than at the distal end of the endoscope. Typically, the output cone of the light exiting the optical fibre 206 does not evenly illuminate the field of view of the camera of the endoscope. In particular, usually the edges of the field of view have poor illumination. As an example, the optical fibre 206 may provide a cone of light with a half angle (denoted 202 in FIG. 2) of 37 degrees, whereas the camera of the endoscope may have a field of view with a half angle of 40 degrees.

The illumination of the surgical site provided by an endoscope could be improved by placing the LED at the distal end of the endoscope. However, it is known that LEDs generate too much heat for this to be a practical option. Furthermore, often an LED would be too large to place at the distal end of the endoscope. For example, an LED might be larger than the endoscope itself, mainly due to the need for heat sinking. As such, for an endoscope that is to be used for surgery, i.e. where the distal end of the endoscope is to be inserted inside a patient's body, it is not acceptable to position an LED at the distal end of the endoscope.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

There is provided an endoscope comprising:
a set of one or more optical fibres connecting a proximal end of the endoscope to a distal end of the endoscope, wherein the set of one or more optical fibres is arranged to receive, at the proximal end of the endoscope, light generated by a light source; and
a phosphor arranged at the distal end of the endoscope to: receive light from the set of one or more optical fibres, and emit light having a different spectrum to the light from the light source.

The endoscope may further comprise a light interface configured to receive the light generated by the light source and provide the light to the set of one or more optical fibres at the proximal end of the endoscope.

The light source may be a laser.

The diameter of the set of one or more optical fibres may be less than 500 μm.

The endoscope may be flexible along at least a portion of its length from the proximal end to the distal end.

The light emitted from the phosphor may have a wider range of wavelengths than the light generated by the light source.

The spectrum of the light generated by the light source may have a peak within a range of wavelengths from 400 nm to 500 nm.

According to a human visual system, the light generated by the light source may be perceived to be blue light and the light emitted from the phosphor may be perceived to be white light.

The endoscope may further comprise a camera, and wherein the phosphor is arranged to emit light over a range of angles that is greater than a range of angles included within a field of view of the camera.

The endoscope may further comprise one or more mirrored surfaces at the distal end of the endoscope, positioned around the phosphor to reflect light that is emitted backwards from the phosphor.

Said one or more mirrored surfaces may form an open cylinder around the phosphor. The one or more mirrored surfaces may comprise:
a first mirrored surface, which is flat and circular, and positioned behind the phosphor, and
one or more other mirrored surfaces forming side walls of the open cylinder around the phosphor.

The first mirrored surface may have a hole in which a portion of the set of one or more optical fibres is positioned.

The set of one or more optical fibres may have just a single optical fibre.

The set of one or more optical fibres may be further arranged to receive at the proximal end of the endoscope, further light from a further light source. The phosphor may be further arranged to: receive the further light from the set of one or more optical fibres, and scatter the further light.

The further light source may be a laser.

The further light may be fluorescence excitation light for fluorescence guided surgery.

The endoscope may further comprise a filter at the distal end of the endoscope, arranged to filter the fluorescence excitation light to attenuate components of the fluorescence excitation light at a fluorescence detection wavelength.

The endoscope may be a surgical endoscope configured to illuminate and image a surgical site during a surgical operation.

The proximal end of the endoscope may be configured to be attached to a distal end of a surgical robot arm.

There is provided a method of operating an endoscope which comprises a set of one or more optical fibres connecting a proximal end of the endoscope to a distal end of the endoscope, the method comprising:
receiving light generated by a light source at the set of one or more optical fibres at the proximal end of the endoscope;
receiving, from the set of one or more optical fibres, light at a phosphor located at the distal end of the endoscope; and
emitting light from the phosphor, wherein the light emitted from the phosphor has a different spectrum to the light from the light source.

There may be provided an endoscope comprising:
a set of one or more optical fibres connecting a proximal end of the endoscope to a distal end of the endoscope, wherein the set of one or more optical fibres is arranged to receive, at the proximal end of the endoscope, first light generated by a first light source and fluorescence excitation light for fluorescence guided surgery generated by a second light source;
a phosphor arranged at the distal end of the endoscope to:
   receive the first light from the set of one or more optical fibres, and emit light having a different spectrum to the first light; and
   receive the fluorescence excitation light from the set of one or more optical
   fibres, and scatter the fluorescence excitation light; and
a filter at the distal end of the endoscope, arranged to filter the fluorescence excitation light to attenuate components of the fluorescence excitation light at a fluorescence detection wavelength.

There may be provided a method of operating an endoscope which comprises a set of one or more optical fibres connecting a proximal end of the endoscope to a distal end of the endoscope, the method comprising:
receiving first light generated by a first light source at the set of one or more optical fibres at the proximal end of the endoscope;
receiving fluorescence excitation light generated by a second light source at the set of one or more optical fibres at the proximal end of the endoscope;
receiving, from the set of one or more optical fibres, the first light at a phosphor located at the distal end of the endoscope;
emitting light from the phosphor, wherein the light emitted from the phosphor has a different spectrum to the first light;
receiving, from the set of one or more optical fibres, the fluorescence excitation light at the phosphor;
scattering the fluorescence excitation light from the phosphor; and
filtering the fluorescence excitation light at the distal end of the endoscope to attenuate components of the fluorescence excitation light at a fluorescence detection wavelength.

The above features may be combined as appropriate, as would be apparent to a skilled person, and may be combined with any of the aspects of the examples described herein.

### BRIEF DESCRIPTION OF THE FIGURES

Examples will now be described in detail with reference to the accompanying drawings in which:

FIG. 1 illustrates an arrangement for coupling light from an LED and a phosphor into an optical fibre for use in a conventional endoscope of the prior art;

FIG. 2 illustrates a distal end of an endoscope of the prior art;

FIG. 3 illustrates a system comprising an endoscope according to examples described herein;

FIG. 4 is a flow chart for a method of operating an endoscope according to examples described herein;

FIG. 5 illustrates a distal end of an endoscope according to examples described herein; and

FIG. 6 illustrates a surgical robotic system comprising an endoscope according to examples described herein.

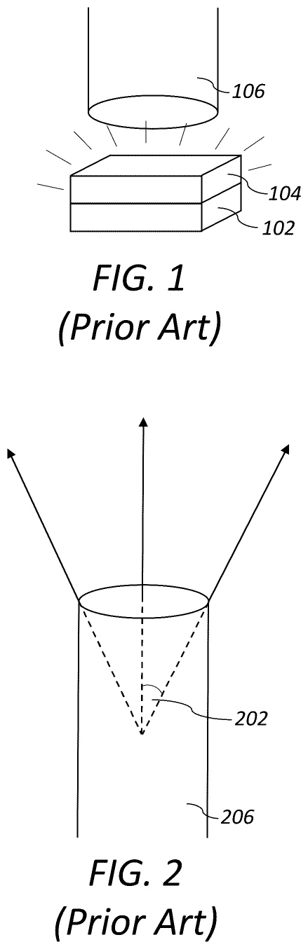

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is presented by way of example to enable a person skilled in the art to make and use the invention. The present invention is not limited to the embodiments described herein and various modifications to the disclosed embodiments will be apparent to those skilled in the art.

In the examples described herein, a phosphor is located at the distal end of the endoscope. A light source provides light to an optical fibre at the proximal end of the endoscope, and the light travels down the optical fibre to the distal end of the endoscope. At least some of the light is absorbed by the phosphor, and the phosphor reemits light from the distal end of the endoscope. The light that is emitted from the phosphor has a different spectrum to the light from the light source. In particular, the light that is emitted from the phosphor has a wider range of wavelengths than the light from the light source. The phosphor will emit the light over a wide range of angles (e.g. the phosphor is a 4 π emitter). In particular, phosphors absorb light and then emit light in a random direction. As such, an arrangement in which the phosphor, but not the light source, is located at the distal end of the endoscope provides for a good, even illumination of the surgical site (e.g. over the whole field of view of a camera of the endoscope), without the light source generating heat at the distal end of the endoscope, and without the light source increasing the size of the distal end of the endoscope.

Furthermore, in examples described herein, rather than using an LED as the light source, a laser may be used as the light source. In contrast to the light emitted from an LED, the light emitted from a laser has low divergence, which allows a laser beam to stay narrow over large distances, and allows the laser light to be focused to a tight spot. Using a laser as the light source means that the light can be directed into a small optical fibre much more efficiently than when an LED is used as the light source. In other words the light from a laser can be coupled into an optical fibre with very high efficiency because the light originates from a small source with low divergence. For example, enough radiant energy can reach the distal end of the endoscope for sufficiently illuminating a surgical site by coupling the light from a laser into an optical fibre having a diameter in a range from 200 μm to 300 μm. As described in the background section above, when an LED is used as the light source, the diameter of a bundle of optical fibres 106 in an endoscope would typically need to be in a range from 3 mm to 5 mm in order for enough radiant energy to reach distal end of the endoscope for sufficiently illuminating a surgical site. Therefore, using a laser as the light source, rather than using an LED, allows the diameter of a set of one or more optical fibres of the endoscope to be reduced by approximately an order of magnitude. Having a smaller diameter for the set of one or more optical fibres means that the optical fibre(s) is (are) small, light and flexible, which are all beneficial characteristics for instruments that are intended to enter a patient's body during minimally invasive surgery.

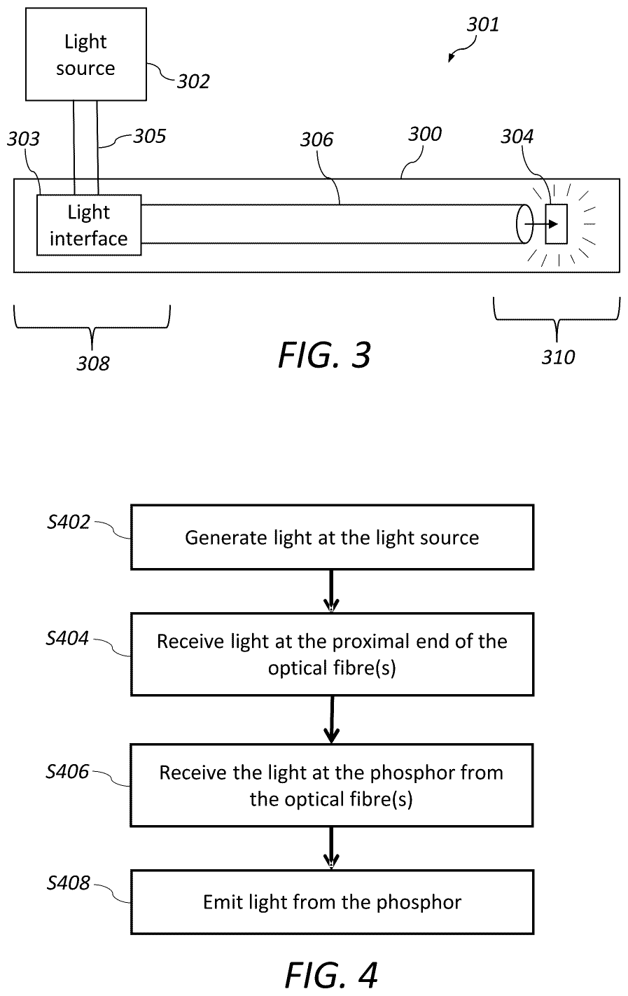

FIG. 3 illustrates a system 301 comprising an endoscope 300 according to examples described herein and a light source 302. The endoscope 300 comprises a light interface 303, a phosphor 304 and a set of one or more optical fibres 306. The light source 302 generates light and provides the light to the light interface 303 of the endoscope 300 using a further set of one or more optical fibres 305. It is noted that it is more efficient to couple light from a single laser into a single optical fibre than to couple light from a single laser into a bundle of fibres because of the gaps that would exist between the fibres in a bundle. As such, in the main examples described herein the set of one or more optical fibres 306 has just a single optical fibre, but it is to be understood that in other examples the set of one or more optical fibres 306 may comprise a plurality of optical fibres, i.e. it may be a bundle of optical fibres, e.g. each carrying light from a different laser. Similarly, in the main examples described herein the further set of one or more optical fibres 305 has just a single optical fibre, but it is to be understood that in other examples the further set of one or more optical fibres 305 may comprise a plurality of optical fibres, i.e. it may be a bundle of optical fibres. The endoscope 300 has a proximal end 308 and a distal end 310. The proximal end 308 of the endoscope 300 is not intended to be inserted into a patient, whereas the distal end 310 of the endoscope 300 is intended to be inserted into a patient, e.g. during surgery. The proximal end 308 of the endoscope 300 comprises the light interface 303 and a proximal end of the optical fibre 306. The distal end 310 of the endoscope 300 comprises the phosphor 304 and a distal end of the optical fibre 306. The optical fibre 306 connects the proximal end 308 of the endoscope 300 to the distal end 310 of the endoscope 300. The light interface 303 is configured to receive the light generated by the light source 302 (via optical fibre 305) and provide the light to the optical fibre 306 at the proximal end of the endoscope 300. Light that is received at the proximal end of the optical fibre 306 can travel through the optical fibre 306 to the distal end of the optical fibre 306.

In conventional systems (e.g. as described in the background section above), a light source is external to the endoscope and light generated by the light source is piped to the endoscope with a thick bundle of optical fibres, e.g. a bundle of optical fibres having a diameter of 5 mm. This thick bundle of optical fibres may comprise thousands of individual optical fibres, each having a diameter of typically 60 microns. A light interface may couple the light between the bundle of optical fibres bringing light to the endoscope and a bundle of optical fibres of the endoscope, which takes the light from the proximal end to the distal end of the endoscope. However, in these conventional systems, the coupling of light between the bundles of optical fibres is poor because the individual optical fibres do not align perfectly. In contrast, in examples described herein the set of optical fibres 305 comprises a single thin optical fibre (e.g. with a diameter of 300 μm) and the set of optical fibres 306 comprises a single thin optical fibre (e.g. with a diameter of 300 μm). The use of a laser as the light source 302 allows single thin optical fibres to be used as the optical fibres 305 and 306, whilst ensuring that enough light reaches the distal end of the endoscope for illuminating the surgical site during minimally invasive surgery. The light interface 303 couples the light from the optical fibre 305 to the optical fibre 306. The coupling of light between single 300 μm optical fibres at the light interface 303 is more efficient than the coupling of light between bundles of 60 μm optical fibres.

In some alternative examples (not shown in the figures), a single set of one or more optical fibres (e.g. a single optical fibre) could take the light from the light source all the way to the phosphor at the distal end of the endoscope. In these alternative examples, the endoscope might not comprise a light interface.

Generally, the light source 302 may be an LED or a laser. In the examples described herein the light source 302 is a laser. As described above, using a laser as the light source provides for a more efficient coupling between the light source 302 and the optical fibre 306 (via the optical fibre 305 and the light interface 303), which in turns allows the diameter of the optical fibre 306 to be reduced by an order of magnitude compared to using an LED as the light source. The diameter of the optical fibre 306 may be less than 500 μm. To give some specific examples, the diameter of the optical fibre 306 may be 200, 300 or 365 μm.

The phosphor 304 could be any suitable phosphor, e.g. cerium (III)-doped yttrium aluminum garnet (YAG:Ce3+) may be used, which is suitable for absorbing blue light and emitting light in a broad range of wavelengths from greenish to reddish, with most of its output in the yellow range of wavelengths. The yellow emission combined with the remaining blue emission gives white light. In another example, the phosphor may be a mixture of blue, green and red-emitting phosphors that absorb violet or ultraviolet light to give a light source of superior colour rendering index. Many other suitable phosphors are available.

FIG. 4 is a flow chart for a method of operating the endoscope 300 in the system 301. In step S402 the light source 302 generates light, e.g. blue laser light. That is, according to a human visual system, the light from the light source may be perceived to be blue light. For example the spectrum of the light from the light source may have a peak in intensity within a range of wavelengths from 400 nm to 500 nm. To give a specific example, the light source 302 may be a laser that generates light with a wavelength of 450 nm. The light generated by the light source 302 is provided to the light interface 303 at the proximal end 308 of the endoscope 300 via the optical fibre 305. The light interface 303 provides the light generated by the light source 302 to the optical fibre 306 at the proximal end 308 of the endoscope 300.

The optical fibre 306 is arranged to receive, at the proximal end 308 of the endoscope 300, light generated by the light source 302. Where the light source 302 is a laser, most of the light generated in step S402 may be received by the optical fibre 306. The light generated by the light source may be focussed (e.g. using a set of one or more lenses) into the optical fibre 305. Focussing the light from a laser onto the tip of the optical fibre is beneficial to get good coupling of the light into the optical fibre. For example, a beam of light generated by a laser may have a diameter of 1000 μm with parallel rays. A lens can focus the beam into a spot that then fits into the optical fibre (which may, for example, have a diameter of 300 μm). The focusing results in rays with a range of converging angles, but these angles are smaller than the acceptance angle of the optical fibre, hence good coupling is achieved. The light is received at the proximal end of the optical fibre 306. The light travels down the optical fibre 306 to the distal end 310 of the endoscope 300.

The phosphor is arranged at the distal end 310 of the endoscope 300 to receive light from the optical fibre 306, i.e. to receive light from the distal end of the optical fibre 306. In step S406 the phosphor 304 receives (at least some of) the light from the optical fibre 306. The light received at the phosphor 304 is absorbed by the phosphor 304. That is, the light received at the phosphor 304 excites the phosphor 304.

In step S408 the phosphor emits light. As described above, the light emitted from the phosphor 304 has a different spectrum to the light from the light source 302. In particular, the light that is emitted from the phosphor 304 in step S408 has a substantially high intensity over a wider range of wavelengths than the light from the light source 302. The light emitted from the phosphor 304 includes light at green and red wavelengths due to the relaxation of the excited states of the phosphor 304. The phosphor 304 does not absorb all of the light that it receives. As such, the light emitted from the phosphor 304 also includes some of the blue light received from the optical fibre, which is scattered rather than absorbed by the phosphor 304. According to a human visual system, the light emitted from the phosphor 304 may be perceived to be white light (e.g. a mixture of red, green and blue light). Furthermore, the phosphor will emit the light over a wide range of angles (e.g. the phosphor may be a 4 π emitter).

The light emitted from the phosphor 304 during a surgical procedure will hit tissue of the patient at the surgical site. Some of the light is absorbed by the tissue, some of the light is scattered within the tissue, and the remainder of the light is reflected back. A light sensor (e.g. a camera) at the proximal (or distal) end of the endoscope receives light reflected back from the tissue. Where the light sensor is implemented at the proximal end of the endoscope, the reflected light may be received at the distal end of the endoscope and passed to the sensor, e.g. through a channel assembly which may combine glass and hollow portions, from the distal end to the proximal end of the endoscope. An image of the tissue may be generated using the reflected light received by the light sensor. The light sensor may be configured to distinguish between light at different wavelengths, e.g. by implementing sensors that receive light a particular ranges of wavelength (e.g. sensors for receiving red light, sensors for receiving green light and sensors for receiving blue light, and possibly sensors for receiving light outside of the visible spectrum), and/or by applying some wavelength-dependent filtering to the light that it receives.

To reiterate some of the description above, in examples described herein, a phosphor which is mounted on the distal tip of an endoscope is excited using light from a blue laser coupled into a small optical fibre. This will produce an intense light at the distal tip of the endoscope without the heat associated with the blue light source being generated at the distal end of the endoscope. Furthermore, it is noted that the phosphor 304 naturally has a high scattering coefficient so not only is the re-emitted green and red light from the phosphor emitted over 4 π radians, but also the residual blue laser light will also be emitted over 4 π radians. As described above, the endoscope comprises a camera (not shown in FIG. 3), and the phosphor is arranged to emit light over a range of angles (e.g. over 4 π radians) that is greater than a range of angles included within a field of view of the camera. The camera may, for example, have an 80 degree field of view. Therefore, during a surgical operation, when the distal end 310 of the endoscope 300 (including the phosphor 304) is inserted inside a patient, the light emitted from the phosphor 304 provides an even white illumination over the full field of view of the camera of the endoscope.

Furthermore, the endoscope may be flexible along at least a portion of its length from the proximal end 308 to the distal end 310. For example, it may be the case that just the distal end 310 of the endoscope 300 is flexible, with the rest of the endoscope being rigid, i.e. having a rigid body. Allowing the endoscope 300 to have a thin optical fibre 306 allows the endoscope to be more manoeuvrable, e.g. allowing for more tilt of the tip (i.e. more tilt of the distal end 310) of the endoscope 300. A thinner optical fibre can be more easily bent, and bent to a greater extent, than a thicker optical fibre, and no light will be lost from the sides of the optical fibre so long as the angle by which the optical fibre bends does not exceed an allowable bend threshold, defined by the properties of the optical fibre.

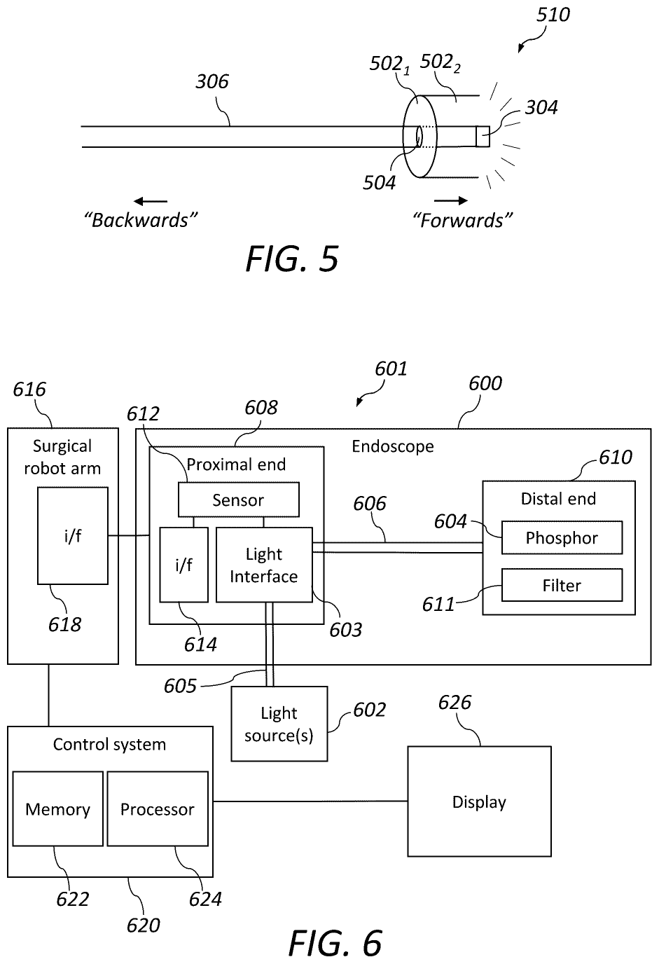

FIG. 5 illustrates an example of a distal end 510 of the endoscope. The distal end 510 comprises the phosphor 304 and the set of one or more optical fibres 306 (which, as mentioned above, is just referred to as an optical fibre herein, but may be a bundle of multiple optical fibres). The distal end 510 further comprises one or more mirrored surfaces 502 positioned around the phosphor 304 to reflect light that is emitted backwards from the phosphor 304. In this way, more of the light that is emitted from the phosphor 304 can be directed to be within a hemisphere generally pointing forwards from the distal end of the endoscope. Here, the term “backwards” refers to a direction from the phosphor 304 towards the distal end of the optical fibre 306 that provides the light to the phosphor 304, and the term “forwards” refers to a direction opposite to the backwards direction. When the endoscope is in a straight position then “backwards” means a direction towards the proximal end of the endoscope, and “forwards” means a direction away from the proximal end of the endoscope, as shown in FIG. 5. It is noted that the endoscope will be arranged so that a camera of the endoscope receives light within a field of view that is wholly within the forwards pointing hemisphere that is illuminated by the light emitted from the phosphor 304.

The phosphor 304 emits light in all directions, i.e. it is a 4 π emitter. By placing the phosphor 304 in an open mirrored chamber the intensity of the forward-emitted light can be increased. For example, the chamber could be cylindrical in shape. In particular, the one or more mirrored surfaces 502 may form an open cylinder around the phosphor 304. An open end of the open cylinder may point forwards from the phosphor 304. That is, the one or more mirrored surfaces 502 may comprise: (i) a first mirrored surface 502₁ forming a base of the open cylinder, which is flat and circular, and positioned behind the phosphor 304 (i.e. positioned at a location that is ‘backwards’ from the position of the phosphor); and (ii) one or more other mirrored surfaces 502₂ forming side walls of the open cylinder around the phosphor 304. To give two examples, the side walls of the open cylinder may be formed as a single continuous surface or as two mirrored surfaces each shaped as half of a cylindrical side wall. The phosphor 304 may be positioned approximately in the centre of the open top surface of the open cylinder. In this way, light that is emitted in a generally forwards direction from the phosphor 304 will not hit one of the mirrored surfaces 502; whereas, light that is emitted in a generally backwards direction from the phosphor 304 will hit (and reflect off) one or more of the mirrored surfaces 502. The light may be reflected one or more times before exiting the mirrored chamber in a generally forwards direction. It is noted that arranging the mirrored surfaces into a cylindrical shape as shown in FIG. 5 would tend to provide a more even emission of light from the endoscope than arranging the mirrored surfaces into a ‘boxed’ or cuboidal shape due to the additional corners in a boxed or cuboidal shape.

As shown in FIG. 5, the first mirrored surface 502₁ has a hole 504 in which a portion of the optical fibre 306 is positioned. This allows the distal end of the optical fibre to be located adjacent to the phosphor 304 so that light that travels through the optical fibre can be provided directly to the phosphor 304. The shape and size of the hole 504 may match the shape and size of the cross-section of the optical fibre 306, so that a portion of the optical fibre 306 can be positioned within the hole 504 without leaving large gaps around the edge of the optical fibre 306. This allows the optical fibre 306 to provide light directly to the phosphor 304 with only a small reduction the reflective area of the mirrored surface 502₁. The optical fibre 306 may pass through the hole 504 (as shown in FIG. 5), or the optical fibre may terminate at the hole 504, i.e. the distal end of the optical fibre may be located at the hole 504.

In another example, there may just be a single mirrored surface. For example, just the first mirrored surface 502₁ may be present, and may be positioned behind the phosphor 304. In this example, the side walls 502₂ would not be present. This may be simpler to manufacture than the example shown in FIG. 5, and may provide a lighter and smaller distal end for the endoscope, but may not be as effective at directing as much of the light emitted from the phosphor 304 in a generally forwards direction.

As described above, the endoscope of the examples described herein may be a surgical endoscope configured to illuminate and image a surgical site during a surgical operation. The endoscope may be a hand-held endoscope, wherein a surgeon or another member of an operating team may hold the endoscope and move it into position manually. Alternatively, the endoscope may be attached to a surgical robot arm and moved by controlling the motion of the surgical robot arm. In particular, the proximal end of the endoscope may comprise an interface by which the endoscope can be connected to a surgical robot arm. In this way, the proximal end of the endoscope can be configured to be attached to a distal end of a surgical robot arm.

FIG. 6 illustrates a surgical robotic system 601 comprising an endoscope 600 and a light source 602. The endoscope 600 comprises a proximal end 608 and a distal end 610 (similar to the proximal end 308 and the distal end 310 of the endoscope 300 described above). The endoscope 600 comprises a set of one or more optical fibres 606 (similar to the optical fibre 306 described above) which connects the proximal end 608 of the endoscope to the distal end 610 of the endoscope. The proximal end 608 comprises a light interface 603, a sensor 612 (e.g. a camera, for capturing images of a surgical site) and a robot interface 614. The light interface 603 is arranged to receive light generated by the light source 602 via a set of one or more optical fibres 605, and to provide the light generated by the light source 602 to the set of one or more optical fibres 606. The set of one or more optical fibres 605 may be referred to as a single optical fibre 605 in this description, but it is to be understood that there may be multiple optical fibres in the set 605; and similarly, the set of one or more optical fibres 606 may be referred to as a single optical fibre 606 in this description, but it is to be understood that there may be multiple optical fibres in the set 606. The light source 602 is similar to the light source 302 described above, and is implemented as a laser (although in other examples may be implemented as an LED). The distal end 610 of the endoscope 600 comprises a phosphor 604 (similar to the phosphor 304 described above). The distal end 610 may also comprise a filter 611, which can be useful when the endoscope is used for fluorescence guided surgery, as described below. The surgical robotic system 601 also comprises a surgical robot arm 616. The surgical robot arm 616 may comprise arm segments (not shown in FIG. 6) which are connected by joints (not shown in FIG. 6) which may be manipulated to control the movement of the surgical robot arm. The surgical robot arm 616 comprises an interface 618 at a distal end of the surgical robot arm. The robot interface 614 of the endoscope 600 is configured to connect to the interface 618 of the surgical robot arm 616, so that the endoscope 600 can be controlled via the surgical robot arm 616. The surgical robotic system 601 also comprises a control system 620 and a display 626. The control system 620 comprises a memory 622 and a processor 624. The control system 620 is coupled to the surgical robot arm 616 and to the display 626. The control system 620 is arranged to control the movement of the surgical robot arm 616 (and thereby control the movement of the endoscope 600 when the endoscope 600 is connected to the surgical robot arm 616) by sending control signals to the surgical robot arm 616 to control the movement of the joints of the surgical robot arm 616. The control system 620 is also arranged to send a signal to the light source 602 (e.g. via the interfaces 618, 614 and 603) to control when the light source 602 is switched on and off.

Light can be received at the distal end 610 of the endoscope 600 from the surgical site. The light received at the distal end 610 of the endoscope 600 may be light that was emitted from the distal end 610 of the endoscope (e.g. from the phosphor 604) and that has been reflected off tissue at the surgical site. The light received at the distal end 610 of the endoscope 600 can pass down a channel assembly (e.g. containing glass and hollow sections) from the distal end 610 to the proximal end 608 where it can be provided to the sensor 612 (e.g. via the light interface 603). The sensor 612 is configured to detect the light to thereby determine image data representing an image of the surgical site. The control system 620 is arranged to receive image data (e.g. for frames of a video sequence) that has been captured by the sensor 612. The control system 620 can process the image data (e.g. to apply image processing such as defective pixel correction, noise reduction, white balancing, etc.) to determine the frames of the video sequence, which can then be sent to the display 626, where they can be displayed, e.g. to a surgeon who is performing the surgery. The memory 622 of the control system 620 may be a non-transitory computer readable medium, and can be used to store image data and/or to store computer executable instructions, which can be executed by the processor 624, e.g. in order to perform the processing steps on the image data to determine the frames of the video sequence to be displayed on the display 626.

In some examples, the light source 602 shown in FIG. 6 might represent multiple light sources. One or multiple optical channels may be used. For example, a separate optical channel may be used for each light source. Alternatively, a single optical channel may be used by multiple light sources if these light sources emit light at different wavelengths (such that the light from the different sources can be distinguished by wavelength division multiplexing). As well as a light source (e.g. a laser) generating blue light as described above, the light sources 602 may comprise a further light source to generate further light which can be received at the proximal end of the optical fibre via the light interface 603. The further light source may be implemented as a laser, which allows the optical fibre 606 to have a small diameter (as described above), although in other examples the further light source may be an LED. The further light source is arranged to generate light with a different peak wavelength to that of the blue light generated by the first light source described above. A camera or other sensor could be included at the proximal end 608) of the endoscope 600 (e.g. in addition to the sensor 612) for detecting light at the wavelength of the light from the further light source. In other examples, the camera(s) (or “sensor(s)”) may be placed at the tip (i.e. the distal end 610) of the endoscope 600. For example, the sensor 612 may be placed at the distal end 610 rather than at the proximal end 608 of the endoscope 600.

The further light generated by the further light source travels down the optical fibre to the distal end of the endoscope. The phosphor is arranged to receive the further light from the optical fibre, and scatter the further light. In this way, the phosphor scatters the further light over a wide range of angles (e.g. over 4 π radians) from the distal end of the endoscope. The further light may be fluorescence excitation light for fluorescence guided surgery. For example, the further light source may generate light with a wavelength in the near-infrared range, e.g. with a wavelength in a range from 700 nm to 850 nm. To give a specific example, the further light source may generate light with a wavelength of 770 nm. Fluorescence guided surgery (FGS) is a medical imaging technique used to detect fluorescently labelled structures during surgery. During FGS, a fluorescent material may be injected into the bloodstream of a patient, and the fluorescent material may collect in some tissues (e.g. cancerous cells) more than others. One example of a fluorescent material is indocyanine green (ICG). The fluorescent material may be excited by shining fluorescence excitation light (which is light at a ‘fluorescence excitation wavelength’ that will particularly excite the fluorescent material) onto the fluorescent material. The fluorescent material then emits light at a ‘fluorescence detection wavelength’ as the fluorescent material relaxes from the excited state. The fluorescence detection wavelength is different to the fluorescence excitation wavelength. In particular, the fluorescence detection wavelength is normally longer than the fluorescence excitation wavelength, i.e. it is normally further into the infra-red spectrum away from the visible wavelengths. For example, the fluorescence detection wavelength may be in a range from 830 nm to 900 nm. To give a specific example, ICG may be excited at 800 nm (i.e. the fluorescence excitation wavelength may be 800 nm) and the fluorescence may be detected at 830-900 nm (i.e. the fluorescence detection wavelength may be in the range from 830 nm to 900 nm). It is noted that the penetration depth of the light into the patient's tissue would be very small (e.g. 100 μm) in the visible wavelengths, but it can reach up to 1-2 cm when an excitation wavelength in the near-infrared region (e.g. in a range from 700 nm to 850 nm) is used. By arranging the phosphor to be at the distal end of the endoscope, an even illumination of the fluorescence excitation light is provided by the endoscope over the surgical site for fluorescence guided surgery.

In some examples, both the white light from the phosphor 604 and the fluorescence excitation light are emitted simultaneously. In some other examples, the white light and the fluorescence excitation light may be emitted one at a time, in an alternating manner. In these other examples, the rate at which the system alternates between emitting white light and emitting fluorescence excitation light, may be fast, e.g. it may switch 60 times per second.

The endoscope may comprise a filter (e.g. filter 611 shown in FIG. 6) at the distal end of the endoscope. The filter may be placed over the front of the phosphor. The filter may be arranged to filter the fluorescence excitation light to attenuate components of the fluorescence excitation light at the fluorescence detection wavelength. In this way, a weak output from the phosphor at the fluorescence detection wavelength can be rejected. This means that the sensor (e.g. sensor 612) can more easily identify the light emitted from the fluorescent material, without as much light coming from the phosphor at the fluorescence detection wavelength. A fluorescence image (i.e. an image showing the light received by the sensor 612 at the fluorescence detection wavelength) and a visible light image (i.e. an image showing the light received in the visible wavelengths can be superimposed on each other (e.g. by the control system 620) and displayed to a surgeon on the display 626.

It is to be understood that the endoscope described herein could be for purposes other than surgery. For example, the endoscope could be used for non-medical uses, e.g. in an industrial field for techniques such as non-destructive testing and hole exploration. For example, the endoscope could be used to view inside a manufactured article such as a car engine, via an inspection port.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. An endoscope comprising:
a set of one or more optical fibres connecting a proximal end of the endoscope to a distal end of the endoscope, wherein the set of one or more optical fibres is arranged to receive, at the proximal end of the endoscope, light generated by a light source; and
a phosphor arranged at the distal end of the endoscope to: receive light from the set of one or more optical fibres, and emit light having a different spectrum to the light from the light source.

2. The endoscope of claim 1 further comprising a light interface configured to receive the light generated by the light source and provide the light to the set of one or more optical fibres at the proximal end of the endoscope.

3. The endoscope of claim 1 wherein the light source is a laser.

4. The endoscope of claim 1 wherein the diameter of the set of one or more optical fibres is less than 500 μm, and wherein the endoscope is flexible along at least a portion of its length from the proximal end to the distal end.

5. The endoscope of claim 1 wherein the spectrum of the light generated by the light source has a peak within a range of wavelengths from 400 nm to 500 nm, and wherein the light emitted from the phosphor has a wider range of wavelengths than the light generated by the light source.

6. The endoscope of claim 1 wherein according to a human visual system, the light generated by the light source is perceived to be blue light and the light emitted from the phosphor is perceived to be white light.

7. The endoscope of claim 1 wherein the endoscope further comprises a camera, and wherein the phosphor is arranged to emit light over a range of angles that is greater than a range of angles included within a field of view of the camera.

8. The endoscope of claim 1 wherein the endoscope further comprises one or more mirrored surfaces at the distal end of the endoscope, positioned around the phosphor to reflect light that is emitted backwards from the phosphor.

9. The endoscope of claim 8 wherein said one or more mirrored surfaces form an open cylinder around the phosphor, with the one or more mirrored surfaces comprising:
a first mirrored surface, which is flat and circular, and positioned behind the phosphor; and
one or more other mirrored surfaces forming side walls of the open cylinder around the phosphor;
wherein the first mirrored surface has a hole in which a portion of the set of one or more optical fibres is positioned.

10. The endoscope of claim 1 wherein the set of one or more optical fibres has just a single optical fibre.

11. The endoscope of claim 1 wherein the set of one or more optical fibres is further arranged to receive at the proximal end of the endoscope, further light from a further light source, and
wherein the phosphor is further arranged to:
receive the further light from the set of one or more optical fibres; and
scatter the further light.

12. The endoscope of claim 11 wherein the further light source is a laser.

13. The endoscope of claim 11 wherein the further light is fluorescence excitation light for fluorescence guided surgery.

14. The endoscope of claim 13 wherein the endoscope further comprises a filter at the distal end of the endoscope, arranged to filter the fluorescence excitation light to attenuate components of the fluorescence excitation light at a fluorescence detection wavelength.

15. The endoscope of claim 13 wherein the second light source generates the fluorescence excitation light with a different peak wavelength to the peak wavelength of the first light generated by first light source.

16. The endoscope of claim 13 wherein the second light source generates the fluorescence excitation light with a wavelength in a range from 700 nm to 850 nm.

17. The endoscope of claim 1 wherein the endoscope is a surgical endoscope configured to illuminate and image a surgical site during a surgical operation.

18. The endoscope of claim 17 wherein the endoscope is further configured to detect fluorescently labelled structures during fluorescence guided surgery.

19. The endoscope of claim 1 wherein the proximal end of the endoscope is configured to be attached to a distal end of a surgical robot arm.

20. A method of operating an endoscope which comprises a set of one or more optical fibres connecting a proximal end of the endoscope to a distal end of the endoscope, the method comprising:
receiving light generated by a light source at the set of one or more optical fibres at the proximal end of the endoscope;
receiving, from the set of one or more optical fibres, light at a phosphor located at the distal end of the endoscope; and
emitting light from the phosphor, wherein the light emitted from the phosphor has a different spectrum to the light from the light source.
